(19) United States
(12) Patent Application Publication
(10) Pub. No.: US 2013/0101540 A1
(43) Pub. Date: Apr. 25, 2013

(54) AQUEOUS DISPERSIONS OF POLYURETHANE AND NANOPARTICLES

(71) Applicants: Serkan Unal, Pittsburgh, PA (US); Yuliya Berezkin, Pittsburgh, PA (US); Peter D. Schmitt, Beaver, PA (US); Karl W. Haider, Wexford, PA (US)

(72) Inventors: Serkan Unal, Pittsburgh, PA (US); Yuliya Berezkin, Pittsburgh, PA (US); Peter D. Schmitt, Beaver, PA (US); Karl W. Haider, Wexford, PA (US); Robert N. Hunt, Steubenville, OH (US)

(73) Assignee: Bayer MaterialScience LLC, Pittsburgh, PA (US)

(21) Appl. No.: 13/278,274

(22) Filed: Oct. 21, 2011

(51) Int. Cl. C08L 75/14; A61Q 19/00; A61K 8/87; A61Q 15/00; B82Y 40/00; B82Y 30/00

(52) U.S. Cl. 424/65; 524/591; 424/78.02; 977/902; 977/926; 977/897

(57) ABSTRACT

The present disclosure is directed to aqueous dispersions comprising polyurethane and nanoparticles, and processes for preparing the aqueous dispersions. The dispersions comprise aqueous polyurethane dispersions having nanoparticles covalently incorporated into the polymer matrix. The dispersions are prepared by reacting isocyanate groups present on the polyurethane chain ends with free amine groups on a hydroxy- or amino-functionalized nanoparticle to covalently attach the polyurethane and the nanoparticle through urethane or urea linkages, respectively.

### FIELD OF THE INVENTION

The present disclosure is directed to compositions and composite materials comprising aqueous dispersions of polyurethane and nanoscale particles. The present disclosure is also directed to processes for producing compositions and composite materials comprising aqueous dispersions of polyurethane and nanoscale particles.

### BACKGROUND OF THE INVENTION

Aqueous polyurethane dispersions (PUDs) may be useful materials for various applications, such as, for example, coatings, adhesives and sealants. See, for example, U.S. Pat. Nos. 6,248,415; 6,284,836; and 6,642,303; which are incorporated by reference herein. PUDs may also find utility in the preparation of film-based articles of manufacture, such as, for example, polyurethane gloves. See, for example, U.S. Pat. No. 7,045,573, which is incorporated by reference herein. PUDs are also relatively environmentally and physiologically friendly owing to a low or zero volatile organic compound (VOC) content, which may facilitate the use of PUDs in personal care products, such as, for example, hair fixatives and skin protection formulations. See, for example, U.S. Pat. Nos. 7,445,770 and 7,452,525, which are incorporated by reference herein.

Nanocomposites comprising blends of organic polymers and inorganic nanoscale particles may exhibit useful properties. For example, nanoparticles physically dispersed in a polymer matrix may impart increased mechanical strength, abrasion resistance, scratch resistance, chemical resistance, thermal stability, radiation stability, and barrier properties, among others. Various organic polymer-inorganic particle nanocomposites and related processes are also known in the art. See, for example, U.S. Patent Application Publication Nos. 2007/0049659 and 2008/0188605; and U.S. Pat. No. 7,189,775, which are incorporated by reference herein.

A need still exists for PUDs incorporating nanoparticles which exhibit good shelf-life stability. In particular, previous systems which incorporate nanoparticles have to be mixed and have the nanoparticles re-dispersed before the PUD can be applied to a substrate.

### SUMMARY OF THE INVENTION

Various embodiments disclosed herein are directed to a process for preparing an aqueous dispersion comprising a polyurethane and nanoparticles. In one embodiment, the process may comprise preparing a polyurethane prepolymer. The polyurethane prepolymer may comprise the reaction product of one or more polyisocyanates and one or more polyols. The prepolymer reaction product may comprise terminal isocyanate groups. The prepolymer reaction product may be reacted with one or more chain-extending compounds to form a polyurethane. At least one of a) the one or more polyols or b) one or more chain-extending compounds may be hydrophilic and/or contain ionic or potentially ionic groups. At least one chain-extending compound may comprise an ionic or potentially ionic group and at least two groups which are reactive to the isocyanate groups on the prepolymer to form the polyurethane. The polyurethane may comprise residual isocyanate chain ends. The residual isocyanate chain ends may be reacted with hydroxy-functional or amino-functional nanoparticles to covalently link the polyurethane to the nanoparticles through a urethane or urea linkage to produce a polyurethane-nanoparticle composite. The composite may be dispersed in an aqueous medium, such as, for example, water or water-containing mixtures.

Other embodiments disclosed herein are directed to aqueous polyurethane dispersions comprising polyurethane covalently linked to nanoparticles through a urethane or urea linkage. The polyurethane may comprise the reaction product of a prepolymer and one or more chain-extending compounds. The prepolymer may comprise the reaction product of one or more polyisocyanates and one or more polyols. At least one chain-extending compound may comprise an ionic or potentially ionic group and at least two groups which are reactive to isocyanate groups.

It is understood that the invention is not limited to the embodiments disclosed in this Summary, and is intended to cover modifications that are within the scope of the invention as defined by the claims.

### BRIEF DESCRIPTION OF THE DRAWINGS

The characteristics and advantages of the present disclosure may be better understood by reference to the accompanying figures, in which:

FIG. 1 is a graph presenting a particle size distribution of platelet-shaped alumina nanoparticles that may find utility in the processes and composites disclosed herein;

FIG. 2 is depicts two (2) graphs presenting particle size distributions of a PUD/alumina nanocomposite blend immediately after preparation and 5 weeks after preparation;

FIG. 3 is depicts two (2) graphs presenting particle size distributions of a covalent PUD/alumina nanocomposite immediately after preparation and 6 months after preparation.

### DETAILED DESCRIPTION OF THE INVENTION

In the present disclosure, including the claims, other than where otherwise indicated, all numbers expressing quantities or characteristics are to be understood as being prefaced and modified in all instances by the term “about.” Accordingly, unless indicated to the contrary, any numerical parameters set forth in the following description may vary depending on the desired properties one seeks to obtain in the compositions and methods according to the present disclosure. At the very least, and not as an attempt to limit the application of the doctrine of equivalents to the scope of the claims, each numerical parameter described in the present description should at least be construed in light of the number of reported significant digits and by applying ordinary rounding techniques.

Also, any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10. Any maximum numerical limitation recited herein is intended to include all lower numerical limitations subsumed therein and any minimum numerical limitation recited herein is intended to include all higher numerical limitations subsumed therein. Accordingly, Applicant(s) reserves the right to amend the present disclosure, including the claims, to expressly recite any sub-range subsumed within the ranges expressly recited herein. The terms “one,” “a,” “an,” or “the” as used herein are intended to include “at least one” or “one or more,” unless otherwise indicated. Thus, the articles “one,” “a,” “an,” and “the” are used herein to refer to one or more than one (i.e., to at least one) of the grammatical object of the article. By way of example, “an element” means one or more elements, and thus, possibly, more than one element is contemplated, and may be employed or used.

Any patent, publication, or other disclosure material, in whole or in part, that is said to be incorporated by reference herein, is incorporated herein in its entirety, but only to the extent that the incorporated material does not conflict with existing definitions, statements, or other disclosure material explicitly set forth in this disclosure. As such, and to the extent necessary, the express disclosure as set forth herein supersedes any conflicting material incorporated herein by reference. Any material, or portion thereof, that is said to be incorporated by reference herein, but which conflicts with existing definitions, statements, or other disclosure material set forth herein is only incorporated to the extent that no conflict arises between that incorporated material and the existing disclosure material.

Embodiments disclosed herein are directed to a process for preparing an aqueous dispersion comprising a polyurethane and nanoparticles. The disclosed embodiments are also directed to the resulting aqueous dispersions. In one embodiment, the disclosed process may comprise preparing a polyurethane prepolymer. The polyurethane prepolymer may comprise the reaction product of one or more polyisocyanates and one or more polyols. The prepolymer reaction product may comprise terminal isocyanate groups. The prepolymer reaction product may be reacted with one or more chain-extending compounds to form a polyurethane. At least one chain-extending compound may comprise an ionic or potentially ionic group and at least two groups which are reactive to the isocyanate groups on the prepolymer to form the polyurethane. The polyurethane may comprise residual isocyanate chain ends. The residual isocyanate chain ends may be reacted with hydroxy- or amino-functional nanoparticles to covalently link the polyurethane to the nanoparticles through urethane or urea linkages to produce a polyurethane-nanoparticle composite. The composite may be dispersed in water.

The disclosed aqueous dispersions may comprise a PUD covalently linked to nanoparticles through urethane or urea linkages. The polyurethane may comprise the reaction product of a prepolymer and one or more chain-extending compounds. The prepolymer may comprise the reaction product of one or more polyisocyanates and one or more polyols. At least one chain-extending compound may comprise an ionic or potentially ionic group and at least two groups which may be reactive to isocyanate groups.

As used herein, the term “polyurethane” refers to any polymer or oligomer comprising urethane (i.e., carbamate) groups, urea groups, or both. Accordingly, the term “polyurethane” as used herein refers collectively to polyurethanes, polyureas, and polymers containing both urethane and urea groups, unless otherwise indicated.

Polyurethanes that may find utility in the disclosed processes and dispersions may comprise prepolymer segments comprising one or more polyisocyanates. As used herein, the term “polyisocyanate” refers to any organic compound which has at least two (2) free isocyanate groups per molecule. Suitable polyisocyanates include organic compounds, such as, for example, diisocyanates having a molecular weight of from about 112 to 1,000. In various embodiments, diisocyanates that may find utility in the disclosed processes and dispersions may have a molecular weight from about 140 to 400. In various embodiments, diisocyanates that may find utility in the disclosed processes and dispersions may be represented by the formula X(NCO)₂, with X representing a divalent aliphatic hydrocarbon radical having from 4 to 18 carbon atoms, a divalent cycloaliphatic hydrocarbon radical having from 5 to 15 carbon atoms, a divalent aromatic hydrocarbon radical having from 6 to 15 carbon atoms or a divalent araliphatic hydrocarbon radical having from 7 to 15 carbon atoms.

Examples of diisocyanates that may find utility in the disclosed processes and dispersions include, but are not limited to, tetramethylene diisocyanate; methylpentamethylene diisocyanate; 1,6-hexamethylene diisocyanate (HDI); dodecamethylene diisocyanate; 1,3-diisocyanatocyclohexane; 1,4-diisocyanatocyclohexane; 1-isocyanato-3,3,5-trimethyl-3-isocyanatomethyl cyclohexane (isophorone diisocyanate or IPDI); 1-isocyanato-3,3,5-trimethyl-5-isocyanatomethyl cyclohexane; 4,4′-diisocyanatobenzene; bis-(4-isocyanatocyclohexyl)-methane (dicyclohexylmethane diisocyanate); 1,3- and 1,4-bis(isocyanatomethyl)-cyclohexane; bis-(4-isocyanato-3-methyl-cyclohexyl)-methane; toluene diisocyanate (TDI); 2,4-diisocyanatotoluene; 2,6-diisocyanatotoluene; hydrogenated toluene diisocyanate; 4,4′-, 2,2′- and 2,4′-diisocyanatodiphenylmethane (MDI monomers); p-xylylene diisocyanate; m-tetramethylxylene diisocyanate; p-isopropylidene diisocyanate; 1,3- and 1,4-diisocyanatomethyl benzene; 1,5-diisocyanato naphthalene, isomers of any of these compounds, and combinations of any of these compounds and/or isomers of any of these compounds.

Mixtures of diisocyanates may find utility in the disclosed processes and dispersions. For example, a mixture of one or more aliphatic diisocyanates and one or more cycloaliphatic diisocyanates may find utility. In certain embodiments, for example, a mixture of hexamethylene diisocyanate and dicyclohexylmethane diisocyanate may find utility.

In addition, polymeric MDI products may find utility in the disclosed processes and dispersions. Polymeric MDI products may be obtained by aniline-formaldehyde condensation followed by phosgenation (raw MDI), which is distilled to at least partially remove MDI monomers to produce the polymeric MDI products.

Higher-functional and/or modified polyisocyanates may also find utility. For example, diisocyanate reaction products including, but not limited to, compounds containing urethane groups, urea groups, allophanate groups, biuret groups, uretdione groups (isocyanate dimer), and/or isocyanurate groups (isocyanate trimer), may find utility in the disclosed processes and dispersions.

Polyurethanes that may find utility in the disclosed processes and dispersions may comprise prepolymer segments comprising one or more polyols. As used herein, the term “polyol” refers to any organic compound which has at least two (2) free hydroxyl groups per molecule. In various embodiments, polyols may be hydrophilic and/or comprise an ionic or potentially ionic group as defined below. In other embodiments, polyols may be hydroxyl-functional nanoparticles.

Ionic or potentially ionic groups that may find utility in polyols may include, for example, ternary ammonium groups, quaternary ammonium groups; groups convertible into such groups; carboxyl groups; carboxylate groups; sulfonic acid groups; sulfonate groups, or combinations of any thereof. In this regard, for example, carboxyl groups and sulfonic acid groups are potentially ionic groups, whereas, carboxylate groups and sulfonate groups are ionic groups in the form of a salt, such as, for example, a sodium salt. Potentially ionic acid groups may be neutralized and converted into ionic groups with base compounds, such as, for example, alkali metal hydroxides, alkali metal hydrogen carbonates, alkali metal carbonates or primary amines, secondary amines or tertiary amines. Exemplary amines include, but are not limited to, diisopropyl ethylamine; triisopropanolamine; trimethyl amine; triethyl amine; triisopropyl amine; tributyl amine; N,N-dimethyl-cyclohexyl amine; N,N-dimethylstearyl amine; N,N-dimethylaniline; N-methylmorpholine; N-ethylmorpholine; N-methylpiperazine; N-methylpyrrolidine; N-methylpiperidine, N,N-dimethyl-ethanol amine, N,N-diethyl-ethanol amine, triethanolamine; N-methyldiethanol amine; dimethylaminopropanol; 2-methoxyethyldimethyl amine; N-hydroxyethylpiperazine; 2-(2-dimethylaminoethoxy)-ethanol or 5-diethylamino-2-pentanone.

In various embodiments, polyols having at least two hydroxyl groups also have number average molecular weights of from 700 to 16,000. Examples of such relatively high molecular weight compounds include polyester polyols, polyether polyols, polyhydroxy polycarbonates, polylactones, polyhydroxy polyacetals, polyhydroxy polyacrylates, polyhydroxy polyamides, polyhydroxy polyester amides, polyhydroxy polyalkadienes, polyhydroxy polythioethers, and combinations of any thereof. These polymeric polyols may comprise from 2 to 8 free hydroxyl groups per molecule.

In certain embodiments, suitable polymeric polyols may comprise two (2) free hydroxyl groups. For example, linear polyester diols may find utility in the disclosed processes and dispersions (branched polyester polyols may also find utility). Polyester polyols may be prepared from aliphatic, cycloaliphatic or aromatic dicarboxylic or polycarboxylic acids and polyhydroxyl alcohols. Examples of dicarboxylic or polycarboxylic acids that may find utility in the preparation of polyester polyols include, but are not limited to, succinic acid; glutaric acid; adipic acid; pimelic acid; suberic acid; azelaic acid; sebacic acid; nonanedicarboxylic acid; decanedicarboxylic acid; terephthalic acid; isophthalic acid; o-phthalic acid; tetrahydrophthalic acid; hexahydrophthalic acid or trimellitic acid, as well as acid anhydrides (such as, for example, o-phthalic acid anhydride, trimellitic acid anhydride or succinic acid anhydride), or combinations of any thereof.

Examples of polyhydroxyl alcohols that may find utility in the preparation of polyester polyols include, but are not limited to, ethanediol; diethylene glycol; triethylene glycol; tetraethylene glycol; 1,2-propanediol; dipropylene glycol; tripropylene glycol; tetrapropylene glycol; 1,3-propanediol; 1,4-butanediol; 1,3-butanediol; 2,3-butanediol; 1,5-pentanediol; 1,6-hexanediol; 2,2-dimethyl-1,3-propanediol (neopentyl glycol); 1,4-dihydroxycyclohexane; 1,4-dimethylolcyclohexane; 1,8-octanediol; 1,10-decanediol; 1,12-dodecanediol and combinations of any thereof. Polyhydroxyl alcohols may also include higher-functional polyols such as trimethylolpropane or glycerol. Cycloaliphatic and/or aromatic dihydroxyl and polyhydroxyl compounds may also be suitable as polyhydroxyl alcohol(s) for the preparation of polyester polyol(s). The corresponding polycarboxylic acid anhydrides or corresponding polycarboxylic acid esters of low alcohols, or mixtures thereof, may also be used in place of the free polycarboxylic acid for the preparation of the polyesters.

The polyester polyols may also be homopolymers or copolymers of lactones, which may be obtained by addition reactions of lactones or lactone mixtures, such as butyrolactone, ε-caprolactone and/or methyl-ε-caprolactone with the suitable difunctional and/or higher-functional starter molecules such as, for example, the low molecular weight polyhydroxyl alcohols mentioned above as structural components for polyester polyols.

Mixtures of one or more dicarboxylic acids and one or more dihydroxyl compounds may find utility in the preparation of polyols for use in the disclosed processes and dispersions. For example, a polyester polyol comprising the reaction products of one (1) dicarboxylic acid and two (2) dihydroxyl compounds may find utility. In certain embodiments, for example, a polyester polyol comprising the reaction products of adipic acid, neopentyl glycol and hexanediol may find utility in the disclosed processes and dispersions.

Hydrophilic polyether polyols and monols may also find utility in the disclosed processes and dispersions. Suitable polyether polyols may be obtained by the reaction of starting compounds which contain reactive hydrogen atoms with alkylene oxides such as ethylene oxide; propylene oxide; butylene oxide; styrene oxide; tetrahydrofuran; epichlorohydrin; or combinations of any of these alkylene oxides. The polyaddition products of these oxides, as well as co-addition and graft products thereof, as well as polyether polyols obtained by condensation of polyhydroxyl alcohols or mixtures thereof and the polyether polyols obtained by alkoxylation of polyhydroxyl alcohols, amines and amino-alcohols, are examples of polyether polyols that may find utility in the disclosed processes and dispersions.

In various embodiments, polyether polyols may comprise the homopolymers, copolymers and graft polymers of propylene oxide and ethylene oxide, which are obtained by addition reactions of the named epoxides with low molecular weight diols or triols, such as have been named above as components for producing polyester polyols, or with higher-functional low molecular weight polyols such as, for example, pentaerythritol or a sugar alcohol, or with water. Polyether monols which may find utility in the disclosed processes and dispersions may comprise the homopolymers, copolymers and graft polymers of propylene oxide and ethylene oxide, which are obtained by addition reactions of the named epoxides with low molecular weight mono-hydroxyl alcohols (e.g., ethanol).

In various embodiments, monohydroxyl-functional polyalkylene oxide compounds may be suitable for use in the disclosed processes and dispersions. In certain embodiments, a monohydroxyl-functional polyether (i.e., a polyether monol) having a number average molecular weight of less than about 3,000, and in some embodiments from 300 to 3,000, based on ethylene oxide or propylene oxide or both, may find utility in the disclosed processes and dispersions. Suitable compounds may include, for example, a polyether monol comprising approximately 85% propylene oxide units and 15% ethylene oxide units (available, for example, as Desmophen® LB-25, Bayer Material Science LLC, Pittsburgh, Pa., USA). In addition, the CARBOWAX SENTRY line of methoxypolyethylene glycols (available from Dow Chemical Company, Midland, Mich., USA), as well as the UCON LB Fluids and UCON 50-HB Fluids (also available from Dow Chemical Company) may find utility. In various embodiments, a monohydroxyl-functional compound may be used in an amount such that the polyalkylene oxide radicals incorporated into the prepolymer via a urethane linkage constitute between 0.1 weight percent to 5 weight percent of the polyurethane prepolymer.

Polyethers modified by vinyl polymers may also find utility in the disclosed processes and dispersions. Products of this kind may be obtained by polymerizing vinyl monomers (e.g. styrene and/or acrylonitrile) in the presence of polyethers (see, for example, U.S. Pat. Nos. 3,383,351; 3,304,273; 3,523,095; 3,110,695 and German Pat. No. 1,152,536, which are incorporated by reference herein).

Polyhydroxy polycarbonates include, for example, the products obtained from the reaction of diols, such as, for example, 1,3-propanediol; 1,4-butanediol; 1,6-hexanediol; diethylene glycol; triethylene glycol or tetraethylene glycol, with diarylcarbonates (e.g. diphenylcarbonate) or phosgene.

Polyhydroxy polythioethers include, for example, the condensation products obtained from thiodiglycol on its own and/or with other glycols, dicarboxylic acids, formaldehyde, aminocarboxylic acids or amino alcohols. The products obtained are either polythio-mixed ethers, polythioether esters or polythioether ester amides, depending on the co-components.

Polyhydroxy polyacetals include, for example, the compounds that can be prepared from aldehydes (e.g. formaldehyde) and glycols, such as, for example diethylene glycol; triethylene glycol; ethoxylated 4,4′-dihydroxy-diphenyldimethylmethane; and 1,6-hexanediol. Polyhydroxy polyacetals that may find utility may also be prepared by the polymerization of cyclic acetals.

Suitable polyhydroxy polyester amides and polyamines include, for example, the predominantly linear condensates obtained from polybasic saturated and unsaturated carboxylic acids or their anhydrides and polyvalent saturated or unsaturated amino-alcohols, diamines, polyamines and mixtures thereof.

Monomers for producing polyhydroxy-functional polyacrylates include, for example, acrylic acid; methacrylic acid; crotonic acid; maleic anhydride; 2-hydroxyethyl acrylate; 2-hydroxyethyl methacrylate; 2-hydroxypropyl acrylate; 2-hydroxypropyl methacrylate; 3-hydroxypropyl acrylate; 3-hydroxypropyl methacrylate; glycidyl acrylate; glycidyl methacrylate; 2-isocyanatoethyl acrylate and 2-isocyanatoethyl methacrylate. If polyhydroxy-functional polyacrylates are used, the dispersion may be cured by free radical polymerization, according to methods well known to those skilled in the art such as exposure to high-energy radiation or water-soluble peroxides or aqueous emulsions of non-water soluble initiators are suitable. These free radical formers may be combined with accelerators in a manner known per se to those skilled in the art.

Suitable polyhydroxy polyalkadienes include, but are not limited to, polybutadienes and polyisoprenes, such as, for example, POLY bd resin from Elf Atochem North America, Philadelphia, Pa. In addition, hydrogenated polyisoprene and hydrogenated polybutadiene, such as, for example, KRATON L-2203 from Shell Chemical, Houston, Tex., and POLYTAIL resins from Mitsubishi Chemical, Tokyo, Japan, may also find utility.

Mixtures of any of the above-described high molecular weight (e.g., having molecular weight of 700-16,000) polyols may find utility in preparing the prepolymers for the processes and dispersions disclosed herein. In addition, polyurethane prepolymers may be prepared by reacting one or more diisocyanates, one or more high molecular weight polyols, and one or more low molecular weight dihydroxyl compounds (e.g., having molecular weight of 50-700). Dihydroxyl compounds that may find utility include, for example, ethylene glycol; diethylene glycol; 1,2-propanediol; 1,3-propanediol; 1,4-butanediol; 1,3-butylene glycol; neopentyl glycol; butyl ethyl propane diol; cyclohexane diol; 1,4-cyclohexane dimethanol; 1,6-hexanediol; bisphenol A (2,2-bis(4-hydroxyphenyl)propane); hydrogenated bisphenol A (2,2-bis(4-hydroxycyclohexyl)propane); and combinations of any thereof.

The dihydroxyl alcohols discussed above in connection with the preparation of polyester polyols or diols and/or polyether polyols, diols or monols which have a relatively low molecular weight may also find utility in preparing polyurethane prepolymers. Additionally, low molecular weight polyesters, including, for example, esters of adipic acid and ethylene glycol may find utility in preparing polyurethane prepolymers. Short-chain homo-addition and co-addition products of ethylene oxide and/or propylene oxide, started on aromatic diols may also find utility. Addition products of alkylene oxides, such as, for example, ethylene oxide and/or propylene oxide, with aromatic dihydroxy compounds or aromatic dicarboxylic acids, such as, for example, hydroquinone, resorcinol, pyrocatechol or 2,2-bis(4-hydroxyphenyl)propane (bisphenol A) may also find utility. The use of low molecular weight diols (e.g., having molecular weight of 50-700) to produce a polyurethane prepolymer may result in a relatively stiff or rigid polymer chain.

Low molecular weight diols may also contain ionic or potentially ionic groups. Exemplary low molecular weight diols containing ionic or potentially ionic groups include those disclosed, for example, in U.S. Pat. No. 3,412,054, incorporated by reference herein. Low molecular weight diols containing ionic or potentially ionic groups that may find utility in the disclosed processes and dispersions include, but are not limited to, dimethylol butanoic acid (DMBA), dimethylol propionic acid (DMBA) and carboxyl-containing caprolactone polyester diol.

Polyurethanes that may find utility in the disclosed processes and dispersions may comprise prepolymer segments that may be chain-extended. The polyurethane prepolymer may be chain extended using at least one chain-extending compound. The chain-extending compound may comprise hydrazine or aliphatic and/or alicyclic primary and/or secondary diamines, such as, for example, methylenediamine; ethylenediamine; propylenediamine; 1,4-butylenediamine; 1,6-hexamethylenediamine; 2-methyl-1,5-pentanediamine (Dytek-A from DuPont); 1-amino-3,3,5-trimethyl-5-aminomethyl cyclohexane (isophorone diamine); piperazine; 1,4-diaminocyclohexane; bis(4-aminocyclohexyl)methane; adipic acid dihydrazide; and combinations of any thereof.

In addition, polyether diamines, which may be prepared by reaction of the corresponding polyether diols with ammonia and/or primary amines, may also find utility. Additional diamines which may find utility include alkylene oxide diamines, such as, for example, 3-{2-[2-(3-aminopropoxy)ethoxy]ethoxy} propylamine (also known as dipropylamine diethyleneglycol or DPA-DEG, available from Tomah Products, Milton, Wis., or available from Air Products, Allentown, Pa. as Ancamine® 1922A), and the other DPA-series ether amines available from Tomah Products, including, for example, dipropylamine propyleneglycol; dipropylamine dipropyleneglycol; dipropylamine tripropyleneglycol; dipropylamine poly(propylene glycol); dipropylamine ethyleneglycol; dipropylamine poly(ethylene glycol); dipropylamine 1,3-propane diol; dipropylamine 2-methyl-1,3-propane diol; dipropylamine 1,4-butane diol; dipropylamine 1,3-butane diol; dipropylamine 1,6-hexane diol; dipropylamine cyclohexane-1,4-dimethanol; and combinations of any thereof.

The chain-extending compound may comprise an ionic or potentially ionic group and at least two groups which are reactive to isocyanate groups on the prepolymer chain ends. As used herein, the term “ionic or potentially ionic group” refers to a chemical group that maintains an ionic charge when incorporated into the polyurethane or a chemical group that is nonionic under certain conditions and ionic under certain other conditions, when incorporated into the polyurethane. For example, in various embodiments, the ionic group or potentially ionic group may comprise a ternary ammonium group; a quaternary ammonium group; a group convertible into such a group; a carboxyl group; a carboxylate group; a sulfonic acid group; a sulfonate group or combinations of any thereof. In this regard, for example, carboxyl groups and sulfonic acid groups are potentially ionic groups, whereas, carboxylate groups and sulfonate groups are ionic groups in the form of a salt, such as, for example, a sodium salt.

The at least partial conversion of the groups convertible into salt groups of the type mentioned may take place before or after incorporation into the polyurethane backbone. The at least partial conversion of the groups convertible into salt groups of the type mentioned may also take place before or during the dispersion of the polyurethane in water. In various embodiments, the chain-extending compound may comprise an ionic or potentially ionic group, such as, for example, N-(2-aminoethyl)-2-aminoethane sulfonic acid (AAS); N-(2-aminoethyl)-2-aminopropionic acid; or the sodium salts thereof. In various embodiments, the chain-extending compound may comprise an ionic or potentially ionic group and two (2) amino groups which are reactive to isocyanate groups on the prepolymer chain ends to form urea groups in the polyurethane backbone.

If the free carboxylic acids or sulfonic acids are incorporated in the polyurethane backbone, then the acids may be neutralized with a neutralizing agent, such as, for example, alkali metal hydroxides, alkali metal hydrogen carbonates, alkali metal carbonates or primary amines, secondary amines or tertiary amines, such as, for example, trialkyl-substituted tertiary amines, before dispersion of the polyurethane resin in water. Exemplary amines include, but are not limited to, diisopropyl ethylamine; triisopropanolamine; trimethyl amine; triethyl amine; triisopropyl amine; tributyl amine; N,N-dimethyl-cyclohexyl amine; N,N-dimethylstearyl amine; N,N-dimethylaniline; N-methylmorpholine; N-ethylmorpholine; N-methylpiperazine; N-methylpyrrolidine; N-methylpiperidine, N,N-dimethyl-ethanol amine, N,N-diethyl-ethanol amine, triethanolamine; N-methyldiethanol amine; dimethylaminopropanol; 2-methoxyethyldimethyl amine; N-hydroxyethylpiperazine; 2-(2-dimethylaminoethoxy)-ethanol or 5-diethylamino-2-pentanone. In certain embodiments, suitable tertiary amines include those which do not contain active hydrogen(s) as determined by the Zerewitinoff test since active hydrogen(s) are capable of reacting with the isocyanate groups of the prepolymer which may undesirably cause gelation, the formation of insoluble particles or chain termination.

The polyurethane component of the disclosed aqueous polyurethane-nanoparticle dispersions may also include compounds which are located at a chain end and which terminate the chain (chain terminators). These chain terminators can be derived, for example, from compounds having the formula:

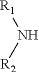

wherein R₁ is an H atom or alkylene radical, optionally having a hydroxyl end and R₂ is an alkylene radical, optionally having a hydroxyl end. For example, suitable compounds include compounds such as monoamines, particularly monosecondary amines, or monoalcohols. Examples include, but are not limited to, methylamine; ethylamine; propylamine; butylamine; octylamine; laurylamine; stearylamine; isononyloxy-propylamine; dimethylamine; diethylamine; dipropylamine; dibutylamine; N-methylaminopropylamine; diethyl(methyl)aminopropylamine; morpholine; piperidine; diethanolamine and suitable substituted derivatives thereof, amide amines of di-primary amines and monocarboxylic acids, monoketimes of di-primary amines, primary/tertiary amines such as N,N-dimethylamino-propylamine and the like. Also suitable are chain terminating alcohols, such as, for example, C₁-C₁₀ or higher alcohols including, methanol, butanol, hexanol, 2-ethylhexyl alcohol, isodecyl alcohol, and the like, and mixtures thereof, as well as amino-alcohols, such as, for example, aminomethylpropanol (AMP).

The disclosed dispersions comprising a polyurethane component set forth herein may also include nanoparticles. As used herein, the terms “nanoscale particle” and “nanoparticle” refer to solid-state particulate material having an average particle size—as determined by the average longest length dimension of the particle—of 20 nanometers to 2000 nanometers. Nanoparticles finding utility in the disclosed processes and dispersions comprise functionalized inorganic nanoparticles or functionalized carbon nanotubes. Examples of functionalized inorganic nanoparticles are metal or metalloid chalcogenide nanoparticles. In various embodiment, the nanoparticles may comprise stoichiometric or non-stoichiometric ceramics including, but not limited to, metal or metalloid oxides, such as, for example, oxides of aluminum, chromium, cobalt, copper, iron, nickel, silicon, titanium and zinc.

The functionalized inorganic nanoparticles comprise a bridging moiety covalently attached to the inorganic nanoparticle. For example, in certain embodiments, the functionalized inorganic nanoparticles may comprise inorganic oxide nanoparticles covalently attached to the bridging moiety, for example, through a covalent bond with an oxygen of a hydroxyl group on the surface of the inorganic oxide nanoparticle, such as, for example, alumina, titanium dioxide, or zinc oxide. The bridging moiety may comprise at least two functional groups; a first functional group configured to covalently bind with an oxygen of a hydroxyl group on the surface of the inorganic oxide nanoparticle, and a second functional groups configured to covalently bind with a free isocyanate group in the polyurethane. The first functional group may comprise, for example, a halogen atom, which may facilitate nucleophilic substitution or nucleophilic addition with a hydroxyl group on the surface of the inorganic oxide, thereby forming a covalent linkage between the nanoparticle and the bridging moiety. The first functional group may also comprise, for example, a carboxylic acid moiety, a sulfonic acid moiety, a phosphonic acid moiety, or a silane moiety. Exemplary processes for functionalizing metal oxide particles are described, for example, in U.S. Pat. Nos. 6,224,846 and 6,846,435, which are incorporated by reference herein.

The second functional group is configured to covalently bind with a free isocyanate group in the polyurethane. In various embodiments, the second functional group may comprise a free amine group or a hydroxyl group, whereas nanoparticles covalently linked to a bridging moiety comprising a free hydroxyl group may be referred to herein as hydroxy-functional nanoparticles. Hydroxy-functional nanoparticles may covalently bind to the polyurethane through a urethane bond whereas amino-functional nanoparticles may covalently bind to the polyurethane through a urea bond to produce a polyurethane-nanoparticle composite, which may alternatively be referred to as a covalent PUD nanocomposite.

In various embodiments, the nanoparticles may comprise functionalized alumina hydrate. Alumina hydrate nanoparticles may comprise hydrated alumina conforming to the formula: Al(OH)ₐO_{b}, where 0<a≦3 and b=(3−a)/2. By way of example, when a=0 the formula corresponds to the alumina formula unit (Al₂O₃). The alumina hydrate nanoparticles may have a water content of 1% to 38% by weight. The alumina hydrate nanoparticles may comprise aluminum hydroxides, such as, for example, aluminum tri-hydroxide (e.g., gibbsite, bayerite), or aluminum oxide hydroxide (e.g., boehmite, diaspore).

In reference to the morphology of inorganic nanoparticles, different morphologies, such as, for example, rod-shaped nanoparticles and platelet-shaped nanoparticles may find utility in the disclosed processes and dispersions. For example, rod-shaped nanoparticles have an aspect ratio, defined as the ratio of the longest length dimension to the next largest length dimension perpendicular to the longest length dimension, of at least 3:1, and in some embodiments at least 6:1. In various embodiments, rod-shaped nanoparticles comprise a longest length dimension of at least 20 nanometers, and in some embodiments of 20 nanometers to 2000 nanometers. In various embodiments, the dimensions perpendicular to the longest length dimension are essentially the same and may be each less than the longest length dimension, and in some embodiments may be less than 20 nanometers.

Platelet-shaped nanoparticles generally comprise opposite major surfaces being generally planar and generally parallel to each other. In various embodiments, the longest length dimension, which is parallel to the opposite major surfaces, may be 20 to 2000 nanometers. The shortest length dimension, which is perpendicular to the opposite major surfaces, may be less than 20 nanometers.

Functionalized carbon nanotubes may comprise functional groups such as hydroxy or amino groups that can be reacted with isocyanates. Exemplary processes for the preparation of hydroxy-functionalized or amino-functionalized carbon nanotubes are described, for example, in copending U.S. Ser. No. 13/050,199 and in Long et al., “Preparation of Urea Functionalized Carbon Nanotubes” Polymer Preprints (ACS, Div of Polym. Chem.) 2009, 50 (1), 453, the entire contents of both of which are hereby incorporated by reference.

The interface between nanoparticles and organic polymers may serve an important role in the properties and performance of polymeric nanocomposite materials. Poor interface interaction between the nanoparticle and the polymer matrix may not only result in undesirable properties, but may also affect the shelf life stability of the composite materials. For example, in the case of PUDs, which are colloidal systems where a high molecular weight polyurethane is dispersed in water and stabilized, the introduction of nanoparticles into these materials may destabilize the polyurethane latex particles dispersed in the water.

The present inventors have developed processes to prepare novel PUD nanocomposites that exhibit improved material properties and performance and also exhibit substantial shelf life stability. The processes involve the direct covalent incorporation of functionalized nanoparticles into the polyurethane matrix. In certain embodiments, the nanoparticles are incorporated into the matrix through covalent attachment to pendant isocyanate groups in the polyurethane polymer backbone. In other embodiments, the nanoparticles are incorporated into the matrix through covalent attachment directly into the polymer backbone. In this manner, nanoparticles may be covalently incorporated into the polymer matrix as polymer chain substituents and/or directly into the polymer chain. The incorporation of nanoparticles may be accomplished, for example, using a solvent process.

In various embodiments, the solvent process may comprise: (1) synthesis of an isocyanate-based polyurethane prepolymer; (2) dissolution of the prepolymer in a solvent; (3) chain extension of the prepolymer with amino-functional chain extenders in the solvent; (4) dispersion of the resulting polyurethane polymer in water by adding water to the solvent solution; and (5) at least partial removal of the solvent by distillation leaving an aqueous PUD.

An exemplary process is described, for example, by D. Dieterich in Houben-Weyl: Methoden der Organischen Chemie, Vol. E20, pp 1670-1681 (1987), which is incorporated by reference herein. In various embodiments, a so-called “acetone process” is used. In this example of a solvent process, the solvent comprises acetone. However, the prepolymer may be dissolved in an any organic solvent that is at least partially water-miscible, has no isocyanate-reactive groups, and is more volatile than water (i.e., is at least partially distillable from a mixed solvent-water solution). Such solvents include, for example, 2-butanone, tetrahydrofuran, dioxane, N-methylformamide, N-methylacetamide, N-methylpyrrolidone, and methylethylketone. Mixtures of solvents may also find utility in the processes disclosed herein.

The polyurethane prepolymer may be dissolved in a solvent. One or more chain-extending compounds (and optional chain-terminating compounds) may be added to the solution and reacted with free isocyanate groups on the polyurethane prepolymer. In certain embodiments, the chain-extending compounds (and optional chain terminators) comprise amine groups which react with the isocyanate groups on the polyurethane prepolymer to form covalent urea linkages in the resulting PUD resin. Sufficient amounts of the optional chain extenders (and/or optional chain terminators) may be used such that the calculated number-average molecular weight (Mₙ) of the resulting polyurethane resin is, for example, between 10,000 and 200,000, and in some embodiments between 10,000 and 100,000.

A non-ionic chain-extending compound may be present in an amount from 15 eq. % to 90 eq. %, and in some embodiments in an amount from 35.0 eq. % to 55 eq. %, based on the residual amount of NCO equivalents present in the polyurethane prepolymer. An ionic or potentially ionic chain-extending compound may be present in an amount from 10 eq. % to 50 eq. %, and in some embodiments in an amount from 25 eq. % to 35 eq. %, based on the residual amount of NCO equivalents present in the polyurethane prepolymer. An optional chain terminator may be present in an amount from 0 to 35 eq. %, based on the residual amount of NCO equivalents present in the polyurethane prepolymer.

The relatively high-molecular weight polyurethane resin may be dispersed in the form of an aqueous dispersion by addition of water to the solution or of the solution to water. In certain embodiments, the resulting PUD comprises polyurethane latex particles of nano-scale dimensions, for example, having mean particle sizes from 20 nm to 2000 nm, and in some embodiments from 50 nm to 300 nm, and in other embodiments from 60 nm to 150 nm. The organic solvent may be partially or wholly removed by distillation, optionally under reduced pressure. In various embodiments, the amount of water in which the polyurethane resin is dispersed is determined in such a way that the aqueous PUDs exhibit a solids content of 20 weight percent to 60 weight percent, and in some embodiments 28 weight percent to 42 weight percent.

Aqueous dispersions comprising inorganic nanoparticles covalently incorporated into PUDs (e.g., covalent PUD/inorganic oxide nanocomposites) may be prepared using a solvent process. In various embodiments, for example, a colloidal suspension of functionalized (e.g., amino-functionalized) inorganic nanoparticles (e.g., metal oxides, such as, for example, aluminum dioxide, titanium dioxide, and zinc oxide) may be added into a solvent process during the chain extension step. In other embodiments, functionalized nanoparticles may be added in a solvent process as free particles not suspended in water or otherwise carried in a fluid medium. In certain embodiments, functionalized nanoparticles (either as part of a colloidal suspension or as free particles) may be added in a solvent process during the initial synthesis of an isocyanate-based polyurethane prepolymer.

In various embodiments, functionalized inorganic nanoparticles may be added to the reaction before or after the addition of chain-extending compounds. In certain embodiments, the presence of residual isocyanate chain-ends in the PUD allows for the covalent incorporation of amino-functionalized inorganic nanoparticles into the polyurethane matrix through urea linkages.

In various embodiments, functionalized inorganic nanoparticles may be added to the prepolymer synthesis reaction. For example, an aqueous dispersion comprising polyurethane and nanoparticles may be prepared by combining at least one polyisocyanate, at least one polyol, and functionalized nanoparticles and reacting these materials to form a polyurethane comprising a nanoparticle covalently linked to the polyurethane. The polyol may comprise an ionic or potentially ionic group (for example, dimethylolpropionic acid) so that the polyurethane formed from the reaction comprises at least one ionic or potentially ionic group. In this manner, a polyurethane comprising at least one ionic or potentially ionic group and nanoparticles covalently linked to the polyurethane may be dispersible in water.

The disclosed nanocomposite dispersions may be processed by conventional processes to obtain films, foils, coatings, finishes and for impregnation of substrates. The nanocomposite dispersions may find utility in the production of films, for example, for the manufacture of polyurethane gloves by a dip process or a coagulation process.

The disclosed nanocomposite dispersions may provide polyurethane films which are resistant to various solvents and chemicals, such as, for example, isopropanol, and which exhibit improved mechanical properties such as tear resistance while maintaining excellent material elongation properties.

The disclosed nanocomposite dispersions may also, depending on their intended use, contain conventional auxiliary agents and additives, such as, for example, cross-linking agents, plasticizers, pigments, defoaming agents, or soft-feel additives.

It is likewise possible to combine the disclosed nanocomposite dispersions with other dispersions, such as, for example, polyacrylate dispersions, natural and synthetic rubber latices such as, for example, nitrile-butadiene rubber (NBR), chloroprene or other homopolymers and copolymers, such as, for example, ethyl vinyl acetate or ethyl vinyl alcohol.

In addition, the disclosed nanocomposite dispersions may find utility in personal care products, such as, for example, skin care products, cosmetics, and deodorants. In certain embodiments, for example, nanoparticulate zinc oxide and/or titanium dioxide may be covalently incorporated into a PUD dispersion which is in turn incorporated into a sunscreen formulation.

The illustrative and non-limiting examples that follow are intended to further describe the embodiments presented herein without restricting their scope. Persons having ordinary skill in the art will appreciate that variations of the Examples are possible within the scope of the invention. All parts and percents are by weight unless otherwise indicated.

### EXAMPLES

### Example 1

An aqueous polyurethane dispersion was prepared using a melt-inverse procedure. 43.21 grams of polyethersulfonate (PES, 96% solids by weight) was added to a 1-liter flask. 34.15 grams of hexamethylene diisocyanate (Desmodur® H, Bayer Material Science LLC, Pittsburgh, Pa., USA) and 124.09 grams of bis-(4-isocyanatocyclohexyl)-methane (dicyclohexylmethane diisocyanate) (Desmodur® W, Bayer Material Science LLC) were added to the flask, followed by addition of 0.04 grams of tin(II) 2-ethylhexanoate (Dabco® T-9, Air Products, Allentown, Pa., USA). The reaction mixture was allowed to exotherm up to 60° C. and was maintained at a reaction temperature of 60° C. for 30 min. Thereafter, 405.09 grams of a polyester polyol of adipic acid, neopentyl glycol and hexanediol (Desmophen® PE 170-HN, Bayer Material Science LLC) and 32.49 grams of a polyether monol comprising approximately 80% percent propylene oxide units and 15% ethylene oxide units (Desmophen® LB-25, Bayer Material Science LLC) was added. The reaction temperature was increased and maintained at 85° C. until theoretical percent isocyanate (% NCO) was reached.

Upon reaching the theoretical isocyanate content, the resulting prepolymer was added into a mixture of 949.65 grams of distilled water and 5.61 grams of Surfynol® 104H surfactant (Air Products), and held at 38° C. with mixing at 600 rpm. After all prepolymer was dispersed in the water, while heating to 48° C., a solution of 3.61 grams of hydrazine hydrate in 3.00 grams of distilled water (64% solids) was added over a 5 minute period. Subsequently, a solution of 24.57 grams of 2-methyl-1,5-pentanediamine (Dytek-A®, Invista, Wilmington, Del., USA) in 49.84 grams of distilled water was added dropwise over a 15 minute period and the reaction was mixed at 48° C. for 1 hour. The final polyurethane product was filtered and the composition and certain properties of the resulting PUD are presented in Table 1. For all Examples in which pH, solids content, viscosity, and particle size are presented, pH was determined using a Fisher Scientific Accument AB 15 pH meter, percentage solids content was determined using a Mettler Toledo HR73 Halogen Moisture Analyzer, viscosity was determined using a Brookfield digital RV Viscometer with spindle #3 at 100 rpm, and particle size was determined using a Horiba Laser Scattering Particle Size Distribution Analyzer LA-910.

### Example 2

An aqueous polyurethane dispersion was prepared using an acetone procedure. 371.59 grams of a polyester polyol of adipic acid, neopentyl glycol and hexanediol (Desmophen® PE 170-HN, Bayer Material Science LLC) and 65.79 grams of hexamethylene diisocyanate (Desmodur® H, Bayer Material Science LLC) were added to a 3 liter flask. The mixture was reacted at 85° C. until theoretical percent isocyanate (% NCO) was reached.

Upon reaching the theoretical isocyanate content, the resulting prepolymer was cooled to 60° C. and dissolved in 788.65 grams of acetone. The prepolymer-acetone solution was mixed for 15 minutes and brought to a temperature of 48° C. Thereafter, a solution of 19.67 grams of sodium N-(2-aminoethyl)-2-aminoethane sulfonate (AAS) and 3.76 grams of ethylene diamine (EDA) in 101.90 grams of distilled water was added to the prepolymer-acetone solution dropwise over a 20 minute period. The resulting combination was mixed for 30 minutes, then 556.82 grams of distilled water at room temperature was added into the reaction flask. The acetone was subsequently distilled off under reduced pressure and 5.46 grams of Kathon™ LX Biocide (1.5% solids) (Rohm & Haas, Philadelphia, Pa., USA) was added. The final polyurethane product was filtered and the composition and certain properties of the resulting PUD are presented in Table 1.

TABLE 1

| | Example 1 | | Example 2 | |
|---|---|---|---|---|
| | wt % | wt % | wt % | wt % |
| Component | (total) | (solids) | (total) | (solids) |
| Desmophen ® PE 170-HN | 24.18 | 18.20 | 33.03 | 30.38 |
| PES | 2.58 | 8.03 | 0.00 | 0.00 |
| Desmophen ® LB-25 | 1.94 | 0.54 | 0.00 | 0.00 |
| Desmodur ® H | 2.04 | 15.43 | 5.85 | 54.43 |
| Desmodur ® W | 7.41 | 36.14 | 0.00 | 0.00 |
| Dabco ® T-9 | 0.002 | — | 0.00 | 0.00 |
| AAS | 0.00 | 0.00 | 1.75 | 6.48 |
| EDA | 0.00 | 0.00 | 0.33 | 8.71 |
| hydrazine hydrate | 0.22 | 5.51 | 0.00 | 0.00 |
| Dytek ®-A | 1.47 | 16.15 | 0.00 | 0.00 |
| water | 59.84 | — | 58.55 | — |
| Surrynol ® 104H | 0.33 | — | 0.00 | — |
| Kathon ™ LX | 0.00 | — | 0.49 | — |

Final PUD Properties

| | | |
|---|---|---|
| % solids | 41.13 | 35.39 |
| viscosity @ 25° C. (cps) | 575 | 370 |
| pH | 7.60 | 6.86 |
| mean particle size (μm) | 0.079 | 0.088 |

### Example 3

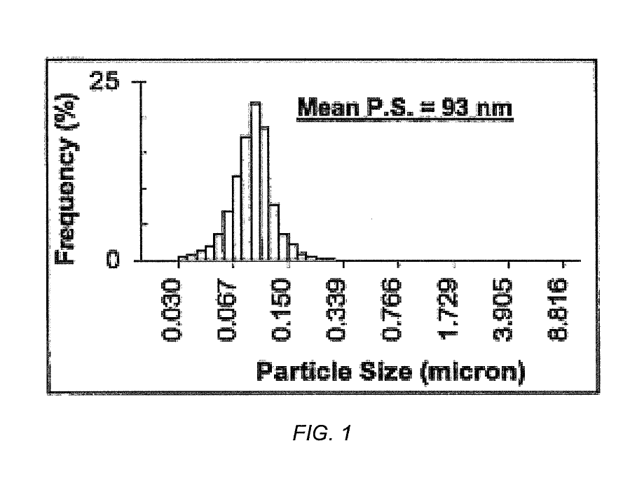

Nanocomposites comprising an aqueous polyurethane dispersion prepared according to Example 1 and a colloidal suspension of alumina nanoparticles were prepared by blending them. Two (2) types of alumina nanoparticles were used. The first type of alumina nanoparticles comprised rod-shaped particles, and the second type of alumina nanoparticles comprised platelet-shaped particles. The respective nanoparticles were suspended in an acidic (pH 3-4) aqueous solution at 15.00% solids concentration by weight. The colloidal suspensions of alumina nanoparticles were sonicated for 2 hours to break up any particle agglomerates. FIG. 1 depicts a typical particle size distribution for the platelet-shaped alumina nanoparticles after sonication and before addition to the PUD.

The nanoparticle suspensions were added dropwise into an aqueous polyurethane dispersion prepared according to Example 1 with mixing at 1200 rpm at room temperature according to the compositions presented in Table 2. After the addition of the colloidal suspension of alumina nanoparticles to the aqueous polyurethane dispersion, the mixture was further mixed at 1000 rpm for 1 hour.

TABLE 2

| | Example | Example | Example | Example |
|---|---|---|---|---|
| | 3a | 3b | 3c | 3d |
| Component | wt % | wt % | wt % | wt % |
| PUD Example 1 | 79.72 | 86.34 | 65.50 | 56.98 |
| (41.13% solids) | | | | |
| rod-shaped alumina | 0.00 | 11.84 | 0.00 | 23.44 |
| nanoparticles | | | | |
| (15.00% solids) | | | | |
| platelet-shaped alumina | 10.93 | 0.00 | 26.94 | 0.00 |
| nanoparticles | | | | |
| (15.00% solids) | | | | |
| water | 9.35 | 1.82 | 7.56 | 19.58 |

Final PUD-nanoparticle
Composite Properties

| | | | | |
|---|---|---|---|---|
| % solids | 35.45 | 32.79 | 29.23 | 24.87 |
| viscosity @ 25° C. | 115 | 90 | 45 | 50 |
| (cps) | | | | |
| pH | 6.95 | 6.84 | 6.90 | 6.61 |
| mean particle size | 0.103 | 4.846 | 0.679 | 17.006 |
| (μm) | (bi-modal) | (bi-modal) | (bi-modal) | (bi-modal) |
| wt % alumina in total | 5.0 | 5.0 | 15.0 | 15.0 |
| resin solids | | | | |

Table 2 presents the compositions and certain properties of blends of PUDs prepared according to Example 1 with rod-shaped alumina nanoparticles or platelet-shaped alumina nanoparticles. Introduction of alumina nanoparticles disrupted the particle size distribution of the base PUD, resulting in bimodal particle size distributions and/or higher mean particle size values (Examples 3a-3d in Table 2 compared with Example 1 in Table 1).

It is believed that the alumina nanoparticles are more stable against agglomeration in acidic solutions. Thus, the change in pH upon addition of the acidic colloidal suspension of nanoparticles into the basic PUDs may have caused increased agglomeration of the nanoparticles in the resulting composites. The introduction of rod-shaped alumina nanoparticles resulted in relatively large mean particle size values as seen in Example 3b and Example 3d in Table 2.

The resulting PUD/alumina nanocomposite blends exhibited phase separation over a 2-3 month period. The limited stability may be due, at least in part, to the bi-modal particle size distributions.

### Example 4

Nanocomposites comprising an aqueous polyurethane dispersion prepared according to Example 2 and a colloidal suspension of alumina nanoparticles were prepared by blending them. Two (2) types of alumina nanoparticles were used. The first type of alumina nanoparticles comprised rod-shaped particles, and the second type of alumina nanoparticles comprised platelet-shaped particles. The respective nanoparticles were suspended in an acidic (pH 3-4) aqueous solution at 15.00% solids concentration by weight. The colloidal suspensions of alumina nanoparticles in water were sonicated for 2 hours to break up any particle agglomerates. The nanoparticle suspensions were added dropwise into an aqueous polyurethane dispersion prepared according to Example 2 with mixing at 1200 rpm at room temperature according to the compositions presented in Table 3. After the addition of the alumina nanoparticle suspension to the aqueous polyurethane dispersion, the mixture was additionally mixed at 1000 rpm for 1 hour.

TABLE 3

| | Example 4a | Example 4b |
|---|---|---|
| Component | wt % | wt % |
| PUD Example 2 (35.39% solids) | 89.45 | 89.45 |
| rod-shaped alumina nanoparticles (15.00% | 0.00 | 10.55 |
| solids) | | |
| platelet-shaped alumina nanoparticles | 10.55 | 0.00 |
| (15.00% solids) | | |
| water | 0.00 | 0.00 |

Final PUD-nanoparticle Composite Properties

| | | |
|---|---|---|
| % solids | 33.56 | 34.45 |
| viscosity @ 25° C. (cps) | 255 | 665 |
| pH | 8.42 | 7.98 |
| mean particle size (μm) | 11.682 | 33.868 |
| | (bi-modal) | (bi-modal) |
| % alumina in total resin solids | 5.0 | 5.0 |

Table 3 presents the compositions and certain properties of blends of PUDs prepared according to Example 2 with rod-shaped alumina nanoparticles or platelet-shaped alumina nanoparticles. Introduction of alumina nanoparticles disrupted the particle size distribution of the base PUD, resulting in bimodal particle size distributions and/or higher mean particle size values (Examples 4a-4b in Table 3 compared with Example 2 in Table 1).

As described in Example 3, it is believed that the alumina nanoparticles are more stable against agglomeration in acidic solutions. Thus, the change in pH upon addition of the acidic colloidal suspension of nanoparticles into the basic PUDs may have caused increased agglomeration of the nanoparticles in the resulting composites. The introduction of alumina nanoparticles also resulted in relatively large mean particle size values as seen in Table 3.

The resulting PUD/alumina nanocomposite blends exhibited phase separation over a 2-3 month period. The limited stability may be due, at least in part, to the bi-modal particle size distributions.

### Example 5

A nanocomposite comprising a commercially available aqueous polyurethane dispersion and a colloidal suspension of alumina nanoparticles was prepared by blending them. The aqueous polyurethane dispersion was Bayhydrol® 110 (35.54% solids) (Bayer Material Science LLC). Two (2) types of alumina nanoparticles were used. The first type of alumina nanoparticles comprised rod-shaped particles, and the second type of alumina nanoparticles comprised platelet-shaped particles. The respective nanoparticles were suspended in water at 15.00% solids concentration by weight. The colloidal suspensions of alumina nanoparticles in water were sonicated for 2 hours to break up any particle agglomerates. The nanoparticle suspensions were added dropwise into the Bayhydrol® 110 aqueous polyurethane dispersion with mixing at 1200 rpm at room temperature according to the compositions presented in Table 4. After the addition of the alumina nanoparticle suspension to the aqueous polyurethane dispersion, the mixture was additionally mixed at 1000 rpm for 1 hour.

TABLE 4

| | Example | Example | | | |
|---|---|---|---|---|---|
| | 5a | 5b | Example 5c | Example 5d | Example 5e |
| Component | wt % | wt % | wt % | wt % | wt % |
| Bayhydrol ® 110 | 100.00 | 85.56 | 77.61 | 70.16 | 65.99 |
| rod-shaped alumina | 0.00 | 0.00 | 9.20 | 0.00 | 23.45 |
| nanoparticles (15.00% solids) | | | | | |
| platelet-shaped alumina | 0.00 | 10.14 | 0.00 | 24.93 | 0.00 |
| nanoparticles (15.00% solids) | | | | | |
| water | 0.00 | 4.30 | 13.19 | 4.91 | 10.56 |

Final PUD-nanoparticle
Composite Properties

| | | | | | |
|---|---|---|---|---|---|
| % solids | 35.54 | 31.42 | 28.62 | 27.25 | 26.92 |
| viscosity @ 25° C. (cps) | 335 | 75 | 50 | 45 | 50 |
| pH | 9.05 | 8.65 | 8.71 | 8.81 | 8.48 |
| mean particle size (μm) | 0.071 | 0.181 | 1.879 | 0.901 | 19.967 |
| | | (bi-modal) | (bi-modal) | | (bi-modal) |
| % alumina in total resin solids | 0.0 | 5.0 | 5.0 | 15.0 | 15.0 |

Table 4 presents the compositions and certain properties of Bayhydrol® 110 blends with rod-shaped alumina nanoparticles or platelet-shaped alumina nanoparticles. Introduction of alumina nanoparticles disrupted the particle size distribution of the base Bayhydrol® 110 PUD resulting in bimodal particle size distributions and/or higher mean particle size values (Examples 5b-5e compared with Example 5a in Table 4).

The resulting PUD/alumina nanocomposite blends exhibited phase separation over a 2-3 month period. The limited stability may be due, at least in part, to the bi-modal particle size distributions.

### Example 6

An aqueous dispersion comprising polyurethane covalently attached to alumina nanoparticles was prepared. The alumina nanoparticles were covalently incorporated into the backbone of the aqueous polyurethane dispersion during the synthesis of the PUD using an acetone process.

336.45 grams of a polyester polyol of adipic acid, neopentyl glycol and hexanediol (Desmophen® PE 170-HN, Bayer Material Science LLC) and 59.57 grams of dicyclohexylmethane diisocyanate (Desmodur® H, Bayer Material Science LLC) were added to a 3 liter flask. The mixture was reacted at 85° C. until theoretical percent isocyanate (% NCO) was reached.

Upon reaching the theoretical isocyanate content, the resulting prepolymer was cooled to 60° C. and dissolved in 788.65 grams of acetone. The prepolymer-acetone solution was mixed for 15 minutes and brought to a temperature of 48° C. Thereafter, a solution of 25.96 grams of sodium N-(2-aminoethyl)-2-aminoethane sulfonate (AAS) and 2.23 grams of ethylene diamine (EDA) in 92.26 grams of distilled water was added to the prepolymer-acetone solution dropwise over a 20 minute period at 48° C. The resulting combination was mixed for 30 minutes at 48° C.

Then 85.40 grams of a colloidal suspension of amino-functionalized platelet-shaped alumina nanoparticles comprising free amine groups (which had been sonicated for 2 hours) was added dropwise into the polyurethane reaction solution at 48° C. and with mixing at greater than 500 rpm. The polyurethane-nanoparticle reaction mixture was mixed for an additional 20 minutes. Then 555.52 grams of distilled water at room temperature was added into the reaction flask. The acetone was subsequently distilled off under reduced pressure and 4.95 grams of Kathon™ LX Biocide (Rohm & Haas) was added. The final aqueous dispersion product comprising polyurethane covalently attached to alumina nanoparticles was filtered and the compositions and certain properties of the resulting PU-alumina nanocomposite dispersion are presented in Table 5 as Example 6a. Examples 6b and 6c in Table 5 were prepared in like manner, wherein Examples 6a, 6b and 6c correspond to 2.5, 5.0 and 10.0 weight percent alumina in total resin solids, respectively.

### Example 7

An aqueous dispersion comprising polyurethane and alumina nanoparticles was prepared using the same procedure described in Example 6; however, a colloidal suspension of platelet-shaped alumina nanoparticles that were not functionalized and which did not comprise free amine groups was used. The colloidal solution of non-functionalized platelet-shaped alumina nanoparticles was added dropwise after chain extension of the prepolymer with AAS and EDA and before dispersion of the acetone solution into water and subsequent distillation. The composition and certain properties of the resulting dispersion are presented in Table 5.

TABLE 5

| | Example 6a | | Example 6b | | Example 6c | | Example 7 | |
|---|---|---|---|---|---|---|---|---|
| Component | wt % | eqs % | wt % | eqs % | wt % | eqs % | wt % | eqs % |
| Desmophen ® PE 170-HN | 28.95 | 30.39 | 23.73 | 30.39 | 23.11 | 30.39 | 27.53 | 30.38 |
| Desmodur ® H | 5.13 | 54.46 | 4.20 | 54.45 | 4.09 | 54.46 | 4.87 | 54.43 |
| AAS | 2.23 | 9.44 | 1.84 | 9.46 | 1.78 | 9.44 | 1.46 | 6.48 |
| EDA | 0.19 | 5.71 | 0.16 | 5.69 | 0.15 | 5.71 | 0.28 | 8.71 |
| amino-functionalized | 7.35 | — | 12.05 | — | 28.15 | — | 0.00 | — |
| platelet-shaped alumina | | | | | | | | |
| nanoparticles (12% solids) | | | | | | | | |
| platelet-shaped alumina | 0.00 | — | 0.00 | — | 0.00 | — | 11.11 | — |
| nanoparticles (15% solids) | | | | | | | | |
| water | 55.73 | — | 57.68 | — | 42.37 | — | 48.79 | — |
| Kathon ™ LX (1.5% solids) | 0.43 | — | 0.35 | — | 0.34 | — | 0.40 | — |

Final PUD-nanoparticle
Composite Properties

| | | | | |
|---|---|---|---|---|
| % solids | 35.20 | 28.41 | 32.35 | 35.58 |
| viscosity @ 25° C. (cps) | 370 | 275 | 60 | 120 |
| pH | 7.14 | 7.61 | 7.60 | 7.05 |
| mean particle size (μm) | 0.292 | 0.451 | 0.808 | 0.329 |

During the preparation of the aqueous dispersions comprising polyurethane covalently attached to alumina nanoparticles, the chain extension level of the polyurethane prepolymer in acetone was approximately 63% (Table 5). This provided a sufficient concentration of free isocyanate chain-ends after the chain extension reactions for the formation of urea bonds with the amino-functionalized alumina nanoparticles. However, experimental studies further showed that the amino-functional nanoparticles could be incorporated into the polyurethane backbone of PUDs having up to 90% levels of chain extension of the polyurethane prepolymer in acetone with diamine chain-extending compounds.

Table 5 presents the compositions and certain properties of aqueous dispersions comprising polyurethane covalently attached to alumina nanoparticles, wherein the resulting aqueous-dispersible nanocomposites contained 2.5 wt %, 5.0 wt % and 10.0 wt % platelet-shaped alumina nanoparticles. The introduction of amino-functional alumina nanoparticles during the PUD synthesis resulted in aqueously-dispersed products with slightly larger mean particle sizes compared to a control PUD sample without nanoparticle loading (Examples 6a, 6b and 6c in Table 5 compared to Example 2 in Table 1). All the covalent PUD/alumina nanocomposites prepared according to Example 6 showed mono-modal particle size distributions, in contrast to the PUD/alumina nanocomposites blends prepared according to Examples 3-5.

Not wishing to be bound by theory, it is believed that the nanocomposites prepared according to Example 6 comprise alumina nanoparticles encapsulated within an outer layer of polyurethane covalently attached to the alumina nanoparticle core. The resulting nanocomposite particles comprise stable aqueous dispersions over a 6 month period.

In order to investigate the effect of covalent attachment of alumina nanoparticles to the polyurethane backbone, a product was synthesized according to Example 7 in which 5.0 wt % non-functionalized platelet-shaped alumina nanoparticles were used. This provided a comparative example to Example 6b, which used 5.0 wt % amino-functionalized platelet-shaped alumina nanoparticles. It should be noted, however, that residual hydroxyl groups may have been present on the surface of the non-functionalized alumina nanoparticles, which could have enabled covalent incorporation of some of the nanoparticles into the polyurethane to a limited extent.

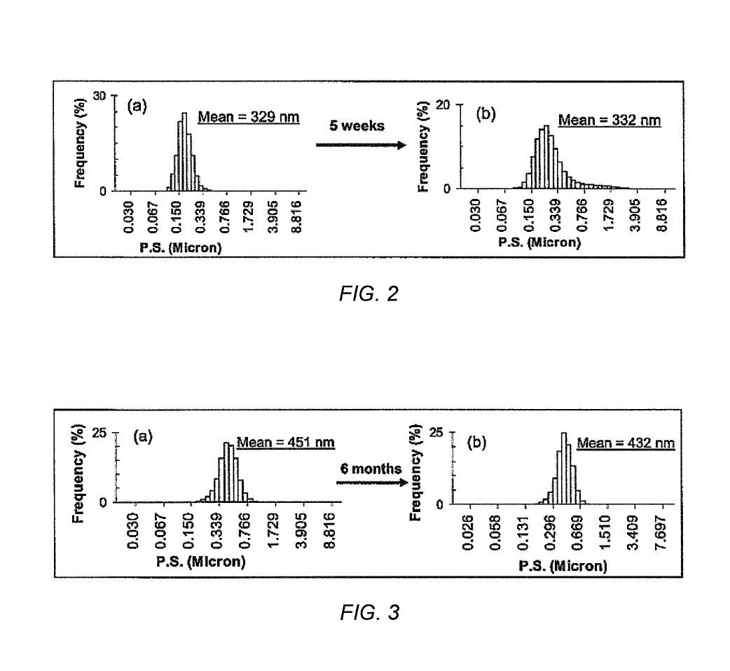

Composites prepared according to Example 7 and Example 6b exhibited mono-modal particle size distributions after the synthesis. However, as shown in FIG. 2, a composite prepared according to Example 7 developed a larger mean particle size and broader size distribution 5 weeks after the synthesis. In contrast, as shown in FIG. 3 the particle size distribution of a composite prepared according to Example 6b did not change over 6 months. Not wishing to be bound by theory, such a change in the particle size distribution of a composite prepared according to Example 7 may be attributed to the migration of encapsulated, non-covalently incorporated alumina nanoparticles from inside the dispersed polyurethane particles into the continuous aqueous phase, where they may agglomerate. Despite the change in the particle size distribution of a composite prepared according to Example 7, no visual phase separation or precipitation was observed over 6 months.

In contrast, as discussed above, the PUD/alumina nanocomposite blends prepared according to Examples 3-5 visibly phase separated over a 2-3 month period. The covalent PUD/alumina nanocomposites prepared according to Example 6 exhibited no phase separation or increase in mean particle size over a 6 month period.

### Example 8

The PUDs and the PUD/alumina nanocomposites blends prepared according to Examples 1 and 3 were subjected to mechanical tensile testing. Tensile tests were performed using an Instron 4444 apparatus. Dog-bone samples with a grip distance of 2.5″ at a crosshead speed of 20 inch/minute were tested and an average of 5-8 samples was reported for each sample. The results of the tensile testing are presented in Table 6.

TABLE 6

| Physical | Exam- | Example | Example | Example | Example |
|---|---|---|---|---|---|
| Properties | ple 1 | 3a | 3b | 3c | 3d |
| 100% Modulus | 520 | 700 | 640 | 620 | 860 |
| 200% Modulus | 830 | 980 | 900 | 910 | 1150 |
| 300% Modulus | 1410 | 1480 | 1310 | 1390 | 1540 |
| Tensile | 4500 | 4060 | 4980 | 4880 | 2940 |
| Strength | | | | | |
| (psi) | | | | | |
| Elongation at | 420 | 450 | 530 | 480 | 480 |
| Break (%) | | | | | |

The elongation values at break improved at all levels of both platelet-shaped and rod-shaped alumina nanoparticles. There was no observable systematic improvement in the tensile strength of PUDs prepared according to Example 1 upon addition of alumina nanoparticles. PUDs prepared according to Example 3b and containing 5 wt % rod-shaped alumina nanoparticles exhibited the most pronounced improvement in most properties, for example, 11% increase in tensile strength, 23% increase in 100% modulus, and 26% increase in elongation at break, compared to control PUD (Example 1).

### Example 9

Films made of PUDs and the PUD/alumina nanocomposites blends prepared according to Examples 2 and 4 were subjected to mechanical tensile testing. Tensile tests were performed using an Instron 4444 apparatus. Dog-bone samples with a grip distance of 2.5″ at a crosshead speed of 20 inch/minute were tested and an average of 5-8 samples was reported for each sample. The results of the tensile testing are presented in Table 7.

TABLE 7

| Physical Properties | Example 2 | Example 4a | Example 4b |
|---|---|---|---|
| 100% Modulus | 220 | 400 | 390 |
| 200% Modulus | 270 | 510 | 470 |
| 300% Modulus | 340 | 660 | 570 |
| 500% Modulus | 610 | 1410 | 1000 |
| Tensile Strength (psi) | 2110 | 2820 | 2920 |
| Elongation at Break (%) | 760 | 620 | 690 |

The addition of 5 wt % platelet-shaped alumina nanoparticles into PUDs prepared according to Example 2 improved the tensile strength by 34%, and improved the modulus at 100% by 82%, while the elongation at break decreased by 18%. The addition of 5 wt % rod-shaped alumina nanoparticles into PUDs prepared according to Example 2 improved the tensile strength by 38%, and the modulus at 100% elongation by 77%, while the elongation at break decreased by 9%.

### Example 10

Films made of Bayhydrol® 110 and the PUD/alumina nanocomposites blends prepared according to Examples 5 were subjected to mechanical tensile testing. Tensile tests were performed using an Instron 4444 apparatus. Dog-bone samples with a grip distance of 2.5″ at a crosshead speed of 20 inch/minute were tested and an average of 5-8 samples was reported for each sample. The results of the tensile testing are presented in Table 8.

TABLE 8

| Physical | Exam- | Example | Example | Example | Example |
|---|---|---|---|---|---|
| Properties | ple 5a | 5b | 5c | 5d | 5e |
| 100% Modulus | 3930 | 3660 | 3940 | 4430 | 5280 |
| Tensile | 5030 | 5910 | 5670 | 5500 | 6310 |
| Strength | | | | | |
| (psi) | | | | | |
| Elongation at | 160 | 210 | 190 | 160 | 160 |
| Break (100%) | | | | | |

The addition of alumina nanoparticles clearly improved the tensile properties of Bayhydrol® 110, while the elongation values at break were retained or improved as well. At a level of 5.0 wt %, platelet-shaped alumina nanoparticles (Example 5b) improved the tensile strength of the coating by 18%, whereas rod-shaped alumina nanoparticles (Example 5c) improved the tensile strength by 13% compared to Bayhydrol 110 control (Example 5a). Elongation values at break also showed 31% and 19% improvement with the addition of 5.0 wt % platelet-shaped and rod-shaped alumina nanoparticles, respectively. Addition of 15.0 wt % platelet-shaped alumina nanoparticles improved the tensile strength by 9% and the modulus at 100% by 13% while showing no effect on elongation at break (Example 5d). Similarly, addition of 15.0 wt % rod-shaped alumina nanoparticles improved the tensile strength by 25% and the modulus at 100% elongation by 34% without affecting the elongation at break (Example 5e).

### Example 11

Films made of covalent PUD/alumina nanocomposites prepared according to Example 6 were subjected to mechanical tensile testing. A comparative nanocomposites prepared according to Example 7 with non-functionalized alumina nanoparticles was also tensile tested. Tensile tests were performed using an Instron 4444 apparatus. Dog-bone samples with a grip distance of 2.5″ at a crosshead speed of 20 inch/minute were tested and an average of 5-8 samples was reported for each sample. The results of the tensile testing are presented in Table 9.

TABLE 9

| | Example | Example | Example | |
|---|---|---|---|---|
| Physical Properties | 6a | 6b | 6c | Example 7 |
| 100% Modulus | 360 | 330 | 420 | 450 |
| 200% Modulus | 460 | 390 | 530 | 560 |
| 300% Modulus | 580 | 460 | 700 | 710 |
| 500% Modulus | 1110 | 780 | 1250 | 1410 |
| Tensile Strength (psi) | 4410 | 4340 | 4460 | 3870 |
| Elongation at Break (%) | 710 | 840 | 745 | 650 |

The tensile strength of coatings from PUDs containing covalently attached platelet-shaped alumina nanoparticles significantly improved compared to the tensile strength of PUDs prepared according to Example 2 without nanoparticles (Table 7). Covalent PUD/alumina nanocomposites containing as low as 2.5 wt % covalently attached platelet-shaped alumina nanoparticles showed >100% improvement in tensile strength without a significant deterioration in elongation at break. The tensile strengths and moduli of the coatings at 5.0 and 10.0 wt % platelet-shaped alumina nanoparticle levels were similarly improved with a slight increase or no decrease in elongation at break. PUDs prepared according to Example 7 and comprising non-functionalized platelet-shaped alumina nanoparticles, showed significantly improved tensile strength and moduli, with a 15% decrease in elongation at break.

### Example 12

An aqueous polyurethane dispersion comprising 0.5 weight percent by solids covalently attached multi-walled carbon nanotubes (MWCNTs) was prepared. Amino-functionalized MWCNTs were and they were covalently incorporated into the backbone of the aqueous polyurethane dispersion during the synthesis of the PUD using an acetone process. 62.99 grams of a polyester polyol of adipic acid, neopentyl glycol and hexanediol (Desmophen® PE 170-HN, Bayer Material Science LLC), 3.22 g mono-hydroxy-functional polyether based on ethylene oxide/propylene oxide (Desmophen® LB 25, Bayer MaterialScience LLC) and 11.42 grams of dicyclohexylmethane diisocyanate (Desmodur® H, Bayer Material Science LLC) were added to a 1 liter flask. The mixture was reacted at 90° C. until theoretical percent isocyanate (% NCO) was reached.

Upon reaching the theoretical isocyanate content, the resulting prepolymer was dissolved in 144.15 grams of acetone and the prepolymer-acetone solution was mixed for 15 minutes and brought to a temperature of 48° C. Thereafter, a solution of 5.07 grams of sodium N-(2-aminoethyl)-2-aminoethane sulfonate (AAS) and 0.47 grams of ethylene diamine (EDA) in 17.27 grams of distilled water was added to the prepolymer-acetone solution dropwise over a 20 minute period at 48° C. The resulting combination was mixed for 30 minutes at 48° C. Then 0.40 grams of amino-functional MWCNTs, (amino-functionalized Baytubes® C 150 P according to the procedure described in Long et al., “Preparation of Urea Functionalized Carbon Nanotubes” Polymer Preprints (ACS, Div of Polym. Chem.) 2009, 50 (1), 453) in 153.11 grams of distilled water (which had been sonicated for 30 minutes) was added dropwise into the polyurethane reaction solution at 48° C. and with mixing at greater than 500 rpm. The polyurethane-nanoparticle reaction mixture was mixed for an additional 10 minutes. Then 63 grams of distilled water at room temperature was added into the reaction flask. The acetone was subsequently distilled off under reduced pressure and 0.92 grams of Kathon™ LX Biocide (1.50 wt % in distilled water) (Rohm & Haas) was added. The final aqueous dispersion product comprising polyurethane covalently attached to MWCNTs was filtered. The final aqueous dispersion product comprising polyurethane (PU) covalently attached to MWCNTs was filtered and the compositions and certain properties of the resulting PU-MWCNT nanocomposite dispersion are presented in Table 10 as Example 12a. Examples 12b was prepared in like manner, wherein 5.0 weight percent covalently attached MWCNTs in total resin solids.

### Example 13

An aqueous polyurethane dispersion comprising 0.5 wt % MWCNTs was prepared using the same procedure described in Example 12; however, MWCNTs (Baytubes® C 150 P) that were not surface functionalized and which did not comprise free amine groups were used. The dispersion of non-functionalized MWCNTs in water nanoparticles was added dropwise after chain extension of the prepolymer with AAS and EDA and before dispersion of the acetone solution into water and subsequent distillation.

### Comparative Example 14

An aqueous polyurethane dispersion containing no MWCNTs was prepared as a comparison. 209.45 grams of a polyester polyol of adipic acid, neopentyl glycol and hexanediol (Desmophen® PE 170-HN, Bayer Material Science LLC), 10.70 g mono-hydroxy-functional polyether based on ethylene oxide/propylene oxide (Desmophen® LB 25, Bayer MaterialScience LLC) and 37.96 grams of dicyclohexylmethane diisocyanate (Desmodur® H, Bayer Material Science LLC) were added to a 3 liter flask. The mixture was reacted at 95° C. until theoretical percent isocyanate (% NCO) was reached.

Upon reaching the theoretical isocyanate content, the resulting prepolymer was dissolved in 479.34 grams of acetone and the prepolymer-acetone solution was mixed for 30 minutes and brought to a temperature of 48° C. Thereafter, a solution of 16.87 grams of sodium N-(2-aminoethyl)-2-aminoethane sulfonate (AAS) and 1.56 grams of ethylene diamine (EDA) in 57.44 grams of distilled water was added to the prepolymer-acetone solution dropwise over a 35 minute period at 48° C. The resulting combination was mixed for 30 minutes at 48° C. Then 426.77 grams of distilled water at room temperature was added into the reaction flask. 0.04 grams of Foamstar I-305 was added and the acetone was subsequently distilled off under reduced pressure. 3.08 grams of Kathon™ LX Biocide (1.50 wt % in distilled water) (Rohm & Haas) was added and the final aqueous dispersion product comprising polyurethane was filtered.

### Shelf Life Stability of Example 12a and 12b Compared to Example 13:

It should be noted that Examples 12a and 12b showed great shelf life stability with insignificant precipitation of MWCNTs over 6 months, whereas MWCNTs precipitated significantly within 10 days after the preparation of Example 13.

Physical Properties of Films Made from Example 12a Compared to Example 14

In addition, the physical properties of films made from Example 12a and Example 14 are compared in the table below. Example 14 showed elongation up to 960% with a tensile strength of 3670 psi at break; whereas Example 12a did not break up to an elongation of 1300%, which was the maximum elongation capability of the Instron machine.

TABLE 10

| Physical Properties | Example 12a | Example 14 |
|---|---|---|
| 100% Modulus | 190 | 250 |
| 200% Modulus | 210 | 290 |
| 300% Modulus | 250 | 330 |
| Tensile Strength (psi) | Film did not break | 3670 |
| Elongation at Break (%) | at 1300% | 960 |
| | elongation | |

Surface Resistivity of Films Made from Example 12b:

Films made from Example 12b, with 5 weight percent covalently attached MWCNTs in the polyurethane were conductive, with a surface resistivity value of 10⁴ ohms/square.

The present invention has been described with reference to certain exemplary, illustrative and non-limiting embodiments. However, it will be recognized by persons having ordinary skill in the art that various substitutions, modifications or combinations of any of the exemplary embodiments (or portions thereof) may be made without departing from the scope of the invention. Thus, the invention is not limited by the description of the exemplary and illustrative embodiments, but rather by the claims.

What is claimed is:

1. A process for preparing an aqueous dispersion comprising polyurethane and nanoparticles, the process comprising:
(1) preparing an isocyanate-functional polyurethane comprising the reaction product of one or more polyisocyanates and one or more polyols;
(2) reacting the isocyanate-functional polyurethane with one or more chain-extending compounds;
(3) reacting the isocyanate-functional polyurethane with functionalized nanoparticles to covalently link the nanoparticles to the polyurethane through a urethane or urea linkage; and
(4) dispersing the polyurethane in an aqueous medium,
wherein at least one of a) the one or more polyols or b) one or more chain-extending compounds are hydrophilic and/or contain ionic or potentially ionic groups;
and wherein steps (2), (3) and (4) may be performed in any order after step (1).

2. The process of claim 1, wherein the prepolymer and the one or more chain-extending compounds are reacted in the presence of at least one partially water-miscible solvent.

3. The process of claim 2, wherein the solvent is selected from the group consisting of acetone, 2-butanone, tetrahydrofuran, dioxane, N-methylformamide, N-methylacetamide, N-methylpyrrolidone, methyl ethyl ketone, and combinations of any thereof.

4. The process of claim 2, further comprising removing the solvent after the polyurethane is dispersed in an aqueous medium.

5. The process of claim 1, wherein the at least one chain-extending compound comprises an ionic or potentially ionic group and at least two groups which are reactive to isocyanate groups.

6. The process of claim 5, wherein the at least one chain-extending compound comprises N-(2-aminoethyl)-2-aminoethane sulfonic acid sodium salt.

7. The process of claim 6, further comprising reacting the prepolymer with one or more additional chain-extending compounds comprising at least two amine groups.

8. The process of claim 7, wherein the additional claim-extending compounds are selected from the group consisting of hydrazine, methylene diamine, ethylene diamine, 2-methyl-1,5-pentane diamine, isophorone diamine, and combinations of any thereof.

9. The process of claim 1, wherein the functionalized nanoparticle comprises an amino-functional nanoscale oxide particle.

10. The process of claim 9, wherein the functionalized nanoparticle comprises an amino-functional alumina nanoscale particle comprising a platelet shaped morphology.

11. The process of claim 9, wherein the functionalized nanoparticle comprises an amino-functional alumina nanoscale particle comprising a rod shaped morphology.

12. The process of claim 1, wherein the functionalized nanoparticle comprises an amino-functional or hydroxy-functional carbon nanotube.

13. The process of claim 1, wherein the one or more polyisocyanates are selected from the group consisting of hexamethylene diisocyanate, isophorone diisocyanate, methylene dicyclohexyl diisocyanate, diphenylmethane diisocyanate, toluene diisocyanate, m-tetramethylxylene diisocyanate, and combinations of any thereof.

14. The process of claim 1, wherein the one or more polyols are selected from the group consisting of polyester polyols, polyether polyols, polyhydroxy polycarbonates, polyhydroxy polyacetals, polyhydroxy polyacrylates, polyhydroxy polyester amides, polyhydroxy polyalkadienes and polyhydroxy polythioethers.

15. The process of claim 14, wherein the one or more polyols comprise:
a polyester polyol comprising the reaction product of adipic acid, neopentyl glycol and hexanediol;
a polyether monol comprising the reaction product of propylene oxide and ethylene oxide; or
a combination thereof.

16. An aqueous polyurethane dispersion comprising:
a polyurethane covalently linked to nanoparticles through urethane or urea linkages, the polyurethane comprising the reaction product of:
a prepolymer comprising the reaction product of one or more polyisocyanates and one or more polyols; and
one or more chain-extending compounds, wherein at least one chain-extending compound comprises an ionic or potentially ionic group.

17. The dispersion of claim 16, wherein the at least one chain-extending compound comprising an ionic or potentially ionic group comprises N-(2-aminoethyl)-2-aminoethane sulfonic acid sodium salt.

18. The dispersion of claim 16, wherein the functionalized nanoparticle comprises an amino-functional nanoscale oxide particle.

19. The dispersion of claim 16, wherein the functionalized nanoparticle comprises an amino-functional alumina nanoscale particle comprising a rod shaped morphology.

20. The dispersion of claim 16, wherein the functionalized nanoparticle comprises an amino-functional or hydroxy-functional carbon nanotube.

21. The dispersion of claim 18, wherein the functionalized nanoparticle comprises an amino-functional alumina nanoscale particle comprising a platelet shaped morphology.

22. The dispersion of claim 16, wherein the one or more polyisocyanates are selected from the group consisting of hexamethylene diisocyanate, isophorone diisocyanate, methylene dicyclohexyl diisocyanate, diphenylmethane diisocyanate, toluene diisocyanate, m-tetramethylxylene diisocyanate, and combinations of any thereof.

23. The dispersion of claim 16, wherein the one or more polyols are selected from the group consisting of polyester polyols, polyether polyols, polyhydroxy polycarbonates, polyhydroxy polyacetals, polyhydroxy polyacrylates, polyhydroxy polyester amides, polyhydroxy polyalkadienes and polyhydroxy polythioethers.

24. The dispersion of claim 23, wherein the one or more polyols comprise:
a polyester polyol comprising the reaction product of adipic acid, neopentyl glycol and hexanediol;
a polyether monol comprising the reaction product of propylene oxide and ethylene oxide; or
a combination thereof.

25. A product comprising the dispersion of claim 16.

26. The product of claim 25 selected from the group selected consisting of films, foils, coatings, finishes, skin care products, sun care products, cosmetics, and deodorants.

27. A process for preparing an aqueous dispersion comprising polyurethane and nanoparticles, the process comprising:
forming a polyurethane prepolymer by
(1) combining one or more polyisocyanates, one or more polyols, and amino-functional nanoparticles, the one or more polyols being hydrophilic and/or comprising an ionic or potentially ionic group;
(2) reacting the one or more polyisocyanates, the one or more polyols, and at least one amine group on the hydroxy-functionalized or amino-functional nanoparticles to form a polyurethane and nanoparticles covalently linked to the polyurethane through a urethane or urea linkage; and
dispersing the polyurethane in an aqueous medium.

28. The process of claim 27, wherein the one or more polyols comprise dimethylolpropionic acid.

29. A process for preparing an aqueous dispersion comprising polyurethane and nanoparticles, the process comprising:
combining one or more polyisocyanates and one or more polyols, the one or more polyols comprising an ionic or potentially ionic group;
reacting the one or more polyisocyanates and the one or more polyols to form a polyurethane, the polyurethane comprising at least one ionic or potentially ionic group, and residual isocyanate chain ends;
reacting the residual isocyanate chain ends with hydroxy-functionalized or amino-functional nanoparticles to covalently link the nanoparticles to the polyurethane through urethane or urea linkages; and
dispersing the polyurethane in an aqueous medium.

30. The process of claim 29, wherein the one or more polyols comprise dimethylolpropionic acid.

31. A product produced according to the process of claim 1.

32. The product of claim 31 selected from the group selected consisting of films, foils, coatings, finishes, skin care products, sun care products, cosmetics, and deodorants.

33. The process of claim 27, wherein at least one chain-extending compound comprises an ionic or potentially ionic group and at least two groups which are reactive to isocyanate groups is combined with the polyisocyanate, one or more polyols and the hydroxy-functional or amino-functional nanoparticles.
